SEQUENCE LISTING (15 sequences)

SEQ ID NO: 1 - DNA, 33 bp, artificial sequence
  bisulfite-treated
gggcgtttttttgcggtcgacgttcggggtgta

SEQ ID NO: 2 - DNA, 15 bp, artificial sequence
  probe oligonucleotide
ggcgtttttttgcgg

SEQ ID NO: 3 - DNA, 14 bp, artificial sequence
  probe oligonucleotide
tcgacgttcggggt

SEQ ID NO: 4 - DNA, 15 bp, artificial sequence
  probe oligonucleotide
ccgcaaaaaaacgcc

SEQ ID NO: 5 - DNA, 14 bp, artificial sequence
  probe oligonucleotide
accccgaacgtcga

SEQ ID NO: 6 - DNA, 15 bp, artificial sequence
  oligonucleotide
tgtggttgatgtttg

SEQ ID NO: 7 - DNA, 17 bp, artificial sequence
  probe oligonucleotide
ggggcgtttttttgcgg

SEQ ID NO: 8 - DNA, 14 bp, artificial sequence
  probe oligonucleotide
caaaaaaacgcccc

SEQ ID NO: 9 - DNA, 34 bp, artificial sequence
  oligonucleotide
ggggcgtttttttgcggtcgacgttcggggtgta

SEQ ID NO: 10 - DNA, 17 bp, artificial sequence
  oligonucleotide
ccccgcaaaaaaacgcc

SEQ ID NO: 11 - DNA, 14 bp, artificial sequence
  oligonucleotide
agctgcaagcccca

SEQ ID NO: 12 - DNA, 31 bp, artificial sequence
  oligonucleotide
accccgaacgtcgaccgcaaaaaaacgcccc

SEQ ID NO: 13 - DNA, 29 bp, artificial sequence
  oligonucleotide
ggcgtttttttgcggtcgacgttcggggt

SEQ ID NO: 14 - DNA, 31 bp, artificial sequence
  oligonucleotide
accccaaacatcaaccacaaaaaaacgcccc

SEQ ID NO: 15 - DNA, 31 bp, artificial sequence
  oligonucleotide
ggggcgtttttttgcggtcgacgttcggggt

(19) United States
(12) Patent Application Publication
(10) Pub. No.: US 2005/0064428 A1
(43) Pub. Date: Mar. 24, 2005

(54) Method for detecting cytosine-methylation patterns by exponential ligation of hybridised probe oligo-nucleotides (mla)

(76) Inventor: Kurt Berlin, Stahnsdorf (DE)

Correspondence Address: KRIEGSMAN & KRIEGSMAN, 665 FRANKLIN STREET, FRAMINGHAM, MA, 01702, US

(21) Appl. No.: 10/501,040

(22) PCT Filed: Jan. 8, 2003
(86) PCT No.: PCT/DE03/00073

(30) Foreign Application Priority Data
Jan. 8, 2002 (DE) 10201138.9

(51) Int. Cl. C12Q 1/68; C12P 19/34

(52) U.S. Cl. 435/6; 435/91.2

(57) ABSTRACT

A method for the detection of cytosine methylation in DNA samples is described, which is comprised of the following steps: First, a genomic DNA sample, which comprises the DNA to be investigated and background DNA, is chemically treated such that all unmethylated cytosine bases are converted to uracil, while the 5-methylcytosine bases remain unchanged. Then the chemically treated DNA sample is amplified with the use of at least 2 primer oligonucleotides as well as a polymerase, whereby the DNA to be investigated is preferred over the background DNA as the template, and in the last step, the amplificates are analyzed and the methylation status in the DNA to be investigated will be concluded from the presence of an amplificate and/or from the analysis of additional positions.

The present invention concerns a method for the detection of cytosine methylation in DNA samples.

The levels of observation that have been well studied in molecular biology according to developments in methods in recent years include the genes themselves, the transcription of these genes into RNA and the proteins forming therefrom. During the course of development of an individual, which gene is turned on and how the activation and inhibition of certain genes in certain cells and tissues are controlled can be correlated with the extent and nature of the methylation of the genes or of the genome. In this regard, pathogenic states are also expressed by a modified methylation pattern of individual genes or of the genome.

5-Methylcytosine is the most frequent covalently modified base in the DNA of eukaryotic cells. For example, it plays a role in the regulation of transcription, in genetic imprinting and in tumorigenesis. The identification of 5-methylcytosine as a component of genetic information is thus of considerable interest. 5-Methylcytosine positions, however, cannot be identified by sequencing, since 5-methylcytosine has the same base-pairing behavior as cytosine. In addition, in the case of a PCR amplification, the epigenetic information which is borne by the 5-methylcytosines is completely lost.

A relatively new method that in the meantime has become the most widely used method for investigating DNA for 5-methylcytosine is based on the specific reaction of bisulfite with cytosine, which, after subsequent alkaline hydrolysis, is converted to uracil, which corresponds in its base-pairing behavior to thymidine. In contrast, 5-methylcytosine is not modified under these conditions. Thus, the original DNA is converted, so that methylcytosine, which originally cannot be distinguished from cytosine by its hybridization behavior, can now be detected by “standard” molecular biology techniques as the only remaining cytosine, for example, by amplification and hybridization or sequencing. All of these techniques are based on base pairing, which is now fully utilized. The prior art which concerns sensitivity is defined by a method that incorporates the DNA to be investigated in an agarose matrix, so that the diffusion and renaturation of the DNA is prevented (bisulfite reacts only on single-stranded DNA) and all precipitation and purification steps are replaced by rapid dialysis (Olek A, Oswald J, Walter J. A modified and improved method for bisulphite based cytosine methylation analysis. Nucleic Acids Res. 1996 Dec. 15;24(24):5064-6). Individual cells can be investigated by this method, which illustrates the potential of the method. Of course, up until now, only individual regions of up to approximately 3000 base pairs long have been investigated; a global investigation of cells for thousands of possible methylation analyses is not possible. Of course, very small fragments of small quantities of sample cannot be reliably analyzed by this method: These are lost despite the protection from diffusion by the matrix.

An overview of other known possibilities for detecting 5-methylcytosines can be derived from the following review article: Rein T, DePamphilis M L, Zorbas H. Identifying 5-methylcytosine and related modifications in DNA genomes. Nucleic Acids Res. 1998 May 15;26(10):2255-64.

The bisulfite technique has been previously applied only in research, with a few exceptions (e.g., Zeschnigk M, Lich C, Buiting K, Dörfler W, Horsthemke B. A single-tube PCR test for the diagnosis of Angelman and Prader-Willi syndrome based an allelic methylation differences at the SNRPN locus. Eur J Hum Genet. 1997 Mar.-Apr.;5(2):94-8). However, short, specific segments of a known gene have always been amplified after a bisulfite treatment and either completely sequenced (Olek A, Walter J. The pre-implantation ontogeny of the H19 methylation imprint. Nat Genet. 1997 November;17(3):275-6) or individual cytosine positions have been detected by a “primer extension reaction” (Gonzalgo ML, Jones P A. Rapid quantitation of methylation differences at specific sites using methylation-sensitive single nucleotide primer extension (Ms-SNuPE). Nucleic Acids Res. 1997 Jun. 15;25(12):2529-31, WO Patent 95-00669) or an enzyme step (Xiong Z, Laird P W. COBRA: a sensitive and quantitative DNA methylation assay. Nucleic Acids Res. 1997 Jun. 15;25(12):25324). Detection by hybridization has also been described (Olek et al., WO 99-28498).

Urea improves the efficiency of bisulfite treatment prior to the sequencing of 5-methylcytosine in genomic DNA (Paulin R, Grigg G W, Davey M W, Piper M. Urea improves efficiency of bisulphite-mediated sequencing of 5-methylcytosine in genomic DNA. Nucleic Acids Res. 1998 Nov. 1;26(21):5009-10).

Other publications which are concerned with the application of the bisulfite technique to the detection of methylation in the case of individual genes are: Grigg G, Clark S. Sequencing 5-methylcytosine residues in genomic DNA. Bioessays. 1994 June;16(6):431-6, 431; Zeschnigk M, Schmitz B, Dittrich B, Buiting K, Horsthemke B, Dorfler W. Imprinted segments in the human genome: different DNA methylation patterns in the Prader-Willi/Angelman syndrome region as determined by the genomic sequencing method. Hum Mol Genet. 1997 March; 6(3):387-95; Feil R, Chariton J, Bird AP, Walter J, Reik W. Methylation analysis on individual chromosomes: improved protocol for bisulphite genomic sequencing. Nucleic Acids Res. 1994 Feb. 25;22(4):695-6; Martin V, Ribieras S, Song-Wang X, Rio M C, Dante R. Genomic sequencing indicates a correlation between DNA hypomethylation in the 5′ region of the pS2 gene and in its expression in human breast cancer cell lines. Gene. 1995 May 19;157(1-2):261-4; WO 9746705, WO 95-15373 and WO 45560.

Another known method is so-called methylation-sensitive PCR (Herman J G, Graff J R, Myohanen S, Nelkin B D, Baylin S B. (1996), Methylation-specific PCR: a novel PCR assay for methylation status of CpG islands. Proc Natl. Acad Sci USA. September 3;93(18):9821-6). For this method, primers are used, which hybridize either only to a sequence that forms by the bisulfite treatment of a DNA which is unmethylated* at the respective position, or, vice versa, primers which bind only to a nucleic acid which forms by the bisulfite treatment of a DNA unmethylated at the respective position. Amplificates can be produced accordingly with these primers, the detection of which in turn supplies indications of the presence of a methylated or unmethylated position in the sample to which the primers bind.
*sic; methylated?-Trans. Note.

A newer method is also the detection of cytosine methylation by means of a Taqman PCR, which has become known as “Methyl Light” (WO 00/70090). It is possible with this method to detect the methylation status of individual positions or a few positions directly in the course of the PCR, so that a subsequent analysis of the products becomes superfluous.

Matrix-assisted laser desorption/ionization mass spectrometry (MALDI-TOF) is a very powerful development for the analysis of biomolecules (Karas M, Hillenkamp F. Laser desorption ionization of proteins with molecular masses exceeding 10,000 daltons. Anal Chem. 1988 Oct. 15;60(20):2299-301). An analyte is embedded in a light-absorbing matrix. The matrix is vaporized by a short laser pulse and the analyte molecule is transported unfragmented into the gas phase. The analyte is ionized by collisions with matrix molecules. An applied voltage accelerates the ions in a field-free flight tube. Ions are accelerated to varying degrees based on their different masses. Smaller ions reach the detector sooner than large ions.

MALDI-TOF spectroscopy is excellently suitable for the analysis of peptides and proteins.

The analysis of nucleic acids is somewhat more difficult (Gut, I. G. and Beck, S. (1995), DNA and Matrix Assisted Laser Desorption Ionization Mass Spectrometry. Molecular Biology: Current Innovations and Future Trends 1: 147-157.) For nucleic acids, the sensitivity is approximately 100 times poorer than for peptides and decreases overproportionally with increasing fragment size. For nucleic acids, which have a backbone with a multiple negative charge, the ionization process through the matrix is basically inefficient.

Genomic DNA is obtained from DNA of cells, tissue or other test samples by standard methods. This standard methodology is found in references such as Fritsch and Maniatis, Molecular Cloning: A Laboratory Manual, 1989.

Methods for the amplification of DNA fragments are found in the prior art. The most frequently used method, the polymerase chain reaction (PCR), is mainly used for the amplification of discrete fragments of genomic DNA with the use of two primers. The above-mentioned method for methylation detection, MSP, also makes use of this method. Other methods for detection of methylation, which are built upon bisulfite-treated DNA, also make use of PCR as an amplification method in order to overcome problems of sensitivity.

Another known method for the exponential amplification of fragments is the ligase chain reaction (LCR). This method is less suitable for the amplification of genomic segments, but is very well suitable, for example, for detection of mutations. A ligation occurs only when two probes hybridize to the template directly adjacent to one another and an erroneous base pairing is not present where these probes bound one another. Like PCR, LCR can be carried out as an exponential amplification, for example, by means of a heat-stable ligase (see, e.g., WO 94/08047). Like PCR, LCR can also be multiplexed. Additional basic patents with respect to LCR are EP 0 320,308 and EP 0 439,182. In the latter, a combination of LCR with a polymerase reaction is described.

Accordingly, a great many methods for methylation analysis are prior art. The present invention, however, will combine in a particularly advantageous manner one of the LCR-like amplification techniques to the detection, in particular, of a small group of CpG positions with identical methylation status.

In a methylation-sensitive PCR, both primers are selected in such a way that they cover for the most part several methylatable positions to be investigated for their methylation status. Only when these, usually 3 or more, positions have an essentially identical methylation status (e.g., all methylated) does a hybridization of the primer to the position involved in the template occur and an amplification can then take the place by means of PCR. If both primers are selected in this way, then it is possible to achieve very high sensitivity of the method. Thus, for example, 1 continuously methylated template can be detected in a background of 10,000 unmethylated templates, since the unmethylated templates are not amplified when appropriately specific primers are used.

A disadvantage of the method, however, is its direct dependence on sequence. It is necessary to thus find precisely those positions which are present co-methylated, in order to achieve a high sensitivity. If the CpG positions are too far removed from one another, then very long primers are necessary, which in turn can be unfavorable for the PCR itself, and also may be disadvantageous also for the sensitivity. The annealing temperature of such primers is also very high. It is also necessary to find another group of co-methylated positions for the generation of a methylation-sensitive PCR product, for the corresponding reverse primer. This is not possible in all cases. Nevertheless, two primers should bind in a methylation-specific manner, since if this does not occur, a sufficient sensitivity cannot be achieved. Therefore, it is meaningful to achieve a specificity that is as high as possible for the binding of two probes or primers in an already coherent group of methylation positions.

This can be achieved by the methylation-sensitive ligation and amplification (MLA) presented here. It is distinguished from an LCR to the extent that the specificity is not essentially influenced by the ligation step itself, as is the case in point mutation analysis. In the case of MLA, a ligation essentially occurs if the two oligonucleotide probes (or primers) that are utilized hybridize adjacent to one another. They hybridize if the methylation status in the genomic sample was either methylated or unmethylated for the two probe positions.

The present invention thus involves a method, which overcomes the disadvantages of the prior art in the field of methylation detection. It can be used for the amplification and for the indirect detection of the methylation status of a group of CpG positions.

This method can be utilized particularly also for the selective amplification of a DNA to be investigated with a specific methylation status in the presence of sequence-homologous background DNA with another methylation status.

To begin with, the terms “DNA to be investigated” as well as “background DNA” will be explained in the sense of this invention on the example of the prior art (MSP).

The DNA to be investigated as well as the otherwise present nucleic acids, which are named “background DNA” in the following, would otherwise be amplified to the same extent, since the primers used cannot distinguish between DNA to be investigated and background DNA. One possibility for differentiating these DNAs results, however, from the different methylation patterns. A current method is methylation-sensitive PCR, abbreviated MSP (Herman J G, Graff J R, Myohanen S, Nelkin B D, Baylin S B. (1996), Methylation-specific PCR: a novel PCR assay for methylation status of CpG islands. Proc Natl Acad Sci USA. September 3;93(18):9821-6). This method is comprised of several sub-steps. First, a bisulfite treatment corresponding to the prior art is carried out, which in turn leads to the fact that all [unmethylated] cytosine bases are converted to uracil, while the methylated cytosine bases (5-methylcytosine) remain unchanged. In the next step, primers are now used, which are completely complementary to a methylated DNA converted with bisulfite, but not to a corresponding DNA which was originally unmethylated. When a PCR is conducted with such a primer, this leads to the circumstance that only the originally methylated DNA is amplified. It is correspondingly possible to use a primer, which in contrast only amplifies the unmethylated DNA. In this manner, if DNA to be analyzed as well as background DNA are present, the DNA fragments to be investigated will be exclusively and selectively produced, as long as they are distinguished from the background DNA with respect to their methylation status in one CpG position.

The prior art is now to infer the methylation status or the presence of a DNA to be investigated from the detection of such a DNA molecule to be investigated, which in turn principally permits a diagnosis, for example, of a tumor disorder in patients, since it is known that, for example, the serum DNA concentration is in part drastically increased in tumor patients. Only the DNA originating from the tumors will then be detected alongside the background DNA. The DNA analysis in other body fluids is comparable in principle.

The prior art is again a method developed by Epigenomics, which amplifies the DNA to be investigated and the background DNA to the same extent after bisulfite treatment and then the former CpG positions that are contained in the fragment are investigated by hybridization techniques, or alternatively by means of minisequencing or other current methods. This has the advantage that one obtains a quantitative pattern with respect to the investigated methylation positions, i.e., it produces the determination of the degree of methylation of a plurality of positions, which makes possible a very precise classification, e.g., in the case of solid tumors. The disadvantage of this method, however, is that it cannot supply accurate information in cases in which the background DNA is present in great excess, since this DNA is amplified along with the DNA to be investigated and both are analyzed in the mixture. This problem does not exist in the analysis of solid tumors, where one can select the material to be investigated in a targeted manner, but it can complicate the analysis of serum DNA, for example.

The object of the present invention is thus to create a method, which overcomes the disadvantages of the prior art.

The object is solved by a method for the detection of cytosine methylation in DNA samples, whereby the following steps are conducted:
a) a genomic DNA sample is treated in such a way that the unmethylated cytosine bases are converted to uracil, while the 5-methylcytosine bases remain unchanged;
b) the chemically treated DNA sample is amplified with the use of at least 2 pairs of essentially complementary probe oligonucleotides as well as a ligase and
c) the amplificates are analyzed and the methylation status in the DNA to be investigated is concluded from the presence of an amplificate.

It is preferred that in the second step, the DNA to be investigated is preferred over the sequence-homologous background DNA as the template.

It is further preferred that the methylation status in the DNA to be investigated is concluded from the analysis of additional positions in the amplificate.

The method according to the invention is particularly preferred, wherein in step b), the probe oligonucleotides hybridize to a template, if the CpG positions which are covered by these in the genomic DNA sample (or the DNA to be investigated) were methylated and wherein the same probe oligonucleotides hybridize essentially to a lesser extent to templates which were completely or partially unmethylated at these positions.

Also, it is most particularly preferred, wherein in step b), the probe oligonucleotides hybridize to a template, if the CpG positions which are covered by these in the genomic DNA sample (or the DNA to be investigated) were unmethylated and wherein the same probe oligonucleotides hybridize essentially to a lesser extent to templates which were completely or partially methylated at these positions.

In addition, it is further particularly preferred that step b) is conducted in detail as follows:
a) the probe oligonucleotides, which hybridized to adjacent positions on the template, are coupled to one another by ligation,
b) the coupled probe oligonucleotides are dehybridized,
c) probe oligonucleotides complementary to the coupled probe oligonucleotides hybridize to the already coupled probe oligonucleotides and are coupled in turn by ligation and
d) the coupled probe oligonucleotides serve as templates for further ligation steps, so that a further propagation of the coupled probe oligonucleotides is produced.

It is also preferred according to the invention that at least one of the probe oligonucleotides bears a phosphate group at the 5′-end.

In addition, it is preferred that at least one of the probe oligonucleotides is provided with a detectable label, particularly provided with a label detectable by fluorescence. It is particularly preferred that at least two probe oligonucleotides are provided with labels, and that these [probes] modify their properties as a function of the distance between them. It is particularly preferred that the probe oligonucleotides bear at least one fluorescent label. It is also preferred that the probe molecules indicate the amplification either by an increase or a decrease in the fluorescence. It is particularly preferred according to the invention that the increase or the decrease in fluorescence is used directly for the analysis and a conclusion of the methylation status of the DNA to be investigated is made from the modified fluorescent signal.

It is particularly preferred that the background DNA is present in 100-fold the concentration in comparison to the DNA to be investigated. It is further preferred that the background DNA is present in 1000× the concentration in comparison to the DNA to be investigated.

It is preferred also according to the invention that the DNA samples are obtained from serum or other body fluids of an individual. It is also preferred according to the invention, that the DNA samples are obtained from cell lines, blood, sputum, stool, urine, serum, cerebrospinal fluid, tissue embedded in paraffin, for example, tissue from eyes, intestine, kidney, brain, heart, prostate, lungs, breast or liver, histological slides and all possible combinations thereof.

A method is further preferred according to the invention, in which step a) is conducted with a bisulfite (=disulfite, hydrogen sulfite). It is preferred here that the chemical treatment is conducted after embedding the DNA in agarose. It is further preferred according to the invention that in the chemical treatment, a reagent that denatures the DNA duplex and/or a radical trap is (are) present.

It is further preferred that the analysis of step c) is conducted by means of hybridization to oligomer arrays, whereby oligomers can be nucleic acids or molecules such as PNAs that are similar in their hybridization properties.

It is also preferred according to the invention that the analysis in step c) is conducted by means of measuring the length of the amplified DNA under investigation, whereby methods for length measurement comprise gel electrophoresis, capillary gel electrophoresis, chromatography (e.g. HPLC), mass spectrometry and other suitable methods.

It is also preferred according to the invention that the analysis according to step c) is conducted by means of sequencing, wherein methods for sequencing comprise the Sanger method, the Maxam-Gilbert method, and other methods such as sequencing by hybridization (SBH).

It is further preferred that a conclusion is made on the presence of a disease or another medical condition of the patient from the methylation status at the different CpG positions investigated.

It is preferred according to the invention that the amplificates themselves are provided with a detectable label for the detection. It is particularly preferred here that the labels are fluorescent labels. It is also preferred here that the labels are radionuclides. It is most particularly preferred according to the invention that the labels are removable mass labels, which are detected in a mass spectrometer. It is also particularly preferred, however, that the amplificates are detected overall in the mass spectrometer and are thus clearly characterized by their mass.

In addition, it is preferred according to the invention that in addition to the probe oligonucleotides, a blocker oligonucleotide is utilized, which preferably binds to the background DNA and prevents the hybridization of the probe oligonucleotides to the background DNA. It is particularly preferred here that two blocker oligonucleotides (or blocker PNAs, generally blocker molecules) that are complementary to one another are used. In addition, it is particularly preferred that the blocker molecules preferably bind to template strands which correspond in their sequence to a DNA that is methylated after treatment according to step a). Or, vice versa, it is also preferred that the blocker molecules preferably bind to template strands, which correspond in their sequence to a DNA that is unmethylated after treatment according to step a). It is also particularly preferred according to the invention that the blocker molecules bind to several CpG positions in the template DNA or also that the blocker molecules bind to several TpG or CpA positions in the template DNA. In addition, it is preferred here according to the invention that the blocker oligonucleotides are modified at their 3′-end and cannot be essentially degraded by a polymerase with nuclease activity.

A method is preferred according to the invention, wherein step b) is conducted in detail as follows:
a) the probe oligonucleotides (probes) hybridize to positions on the template strand in such a way that a gap of at least one base remains between the 3′-end of the first probe and the 5′-end of the second probe,
b) the 3′-end of the first probe is extended by a polymerase reaction, wherein nucleotides complementary to the template strand are incorporated,
c) the elongated first probe is coupled by ligation to the elongated second probe,
d) the coupled probe oligonucleotides are dehybridized,
e) probe oligonucleotides complementary to the coupled probe oligonucleotides hybridize to the already coupled probe oligonucleotides and are coupled in turn by ligation and
f) the coupled probe oligonucleotides serve as templates for further ligation steps, so that a further propagation of the coupled probe oligonucleotides is produced. It is preferred here that step e) is also conducted analogously to steps a)-c). It is preferred here that a heat-stable polymerase is used.

It is also particularly preferred according to the invention that a heat-stable ligase is used.

In addition, it is preferred according to the invention that several sets of oligonucleotide probes are utilized for several groups of methylation positions and thus a multiplexing of the assay is achieved.

The subject of the present invention is also the use of a method according to the invention for the diagnosis and/or prognosis of adverse events for patients or individuals, whereby these adverse events belong to at least one of the following categories: undesired drug interactions; cancer diseases; CNS malfunctions, damage or disease; symptoms of aggression or behavioral disturbances; clinical, psychological and social consequences of brain damage; psychotic disturbances and personality disorders; dementia and/or associated syndromes; cardiovascular disease, malfunction and damage; malfunction, damage or disease of the gastrointestinal tract; malfunction, damage or disease of the respiratory system; lesion, inflammation, infection, immunity and/or convalescence; malfunction, damage or disease of the body as a consequence of an abnormality in the development process; malfunction, damage or disorder of the skin, the muscles, the connective tissue or the bones; endocrine and metabolic malfunction, damage or disease; headaches or sexual malfunction. The use of a method according to the invention is also preferred for distinguishing cell types or tissues or for investigating cell differentiation.

The subject of the present invention is also a kit comprised of a reagent containing bisulfite, labeled oligonucleotide probes, a preferably heat-stable ligase and buffers, as well as, optionally, instructions for conducting an assay according to the invention.

The object according to the invention, of offering a sensitive method for methylation analysis, which overcomes the disadvantages of the prior art, is solved by the fact that a method is created for the detection of cytosine methylation in DNA samples in which the following steps are conducted:
1. A genomic DNA sample is treated in such a way that the unmethylated cytosine bases are converted to uracil, while the 5-methylcytosine bases remain unchanged,
2. the chemically treated DNA sample is amplified with the use of at least 2 pairs of essentially complementary probe oligonucleotides as well as a ligase, and
3. the amplificates are analyzed and the methylation status in the DNA to be investigated is concluded from the presence of an amplificate.

It is preferred according to the invention that in the second step, the DNA to be investigated is preferred over the background DNA as the template.

In addition, it is preferred that the methylation status in the DNA to be investigated is concluded from the analysis of additional positions in the amplificate.

The 2nd step of the method is most preferably conducted as follows:
a) the probe oligonucleotides hybridize to the template, if the CpG positions which are covered by these in the genomic DNA sample (or the DNA to be investigated) were methylated and the hybridization of these probe oligonucleotides occurs to a basically lesser extent, to templates which were completely or partially unmethylated at these positions,
b) the probe oligonucleotides which hybridized to adjacent positions on the template, are coupled together by ligation,
c) the coupled probe oligonucleotides are dehybridized,
d) probe oligonucleotides complementary to the coupled probe oligonucleotides hybridize to the already coupled probe oligonucleotides and are coupled in turn by ligation and
e) the coupled probe oligonucleotides serve as templates for further ligation steps, so that an exponential propagation of the coupled probe oligonucleotides is produced.

It is also preferred to conduct the 2nd method step as follows:
a) the probe oligonucleotides hybridize to the template, if the CpG positions which are covered by these in the genomic DNA sample (or the DNA to be investigated) were unmethylated and the hybridization of the probe oligonucleotides occurs to a basically lesser extent, to templates which were completely or partially methylated at these positions,
b) the probe oligonucleotides, which hybridized to adjacent positions on the template, are coupled together by ligation,
c) the coupled probe oligonucleotides are dehybridized,
d) probe oligonucleotides complementary to the coupled probe oligonucleotides hybridize to the already coupled probe oligonucleotides and are coupled in turn by ligation and
e) the coupled probe oligonucleotides serve as templates for further ligation steps, so that an exponential propagation of the coupled probe oligonucleotides is produced.

In summary, it should be emphasized that the methylation-sensitive step is the adjacent methylation-sensitive (on the corresponding bisulfite-treated DNA) hybridization of two probe oligonucleotides in step a). If a ligation has occurred, then an exponential amplification of these coupled oligonucleotides results.

Therefore, this ligation can occur, if one of the probe oligonucleotides (one in each essentially complementary pair) bears a terminal phosphate group. Otherwise, this group must be introduced in a separate phosphorylation step.

It is preferred according to the invention that the DNA samples are obtained from serum or other body fluids of an individual.

It is additionally preferred according to the invention, that the DNA samples are obtained from cell lines, blood, sputum, stool, urine, serum, cerebrospinal fluid, tissue embedded in paraffin, for example, tissue from eyes, intestine, kidney, brain, heart, prostate, lungs, breast or liver, histological slides and all possible combinations thereof.

It is again preferred that a conclusion is made on the presence of a disease or another medical condition of the patient from the methylation degree of the different CpG positions investigated.

It is most particularly preferred according to the invention that the chemical treatment is conducted with a bisulfite (=disulfite, hydrogen sulfite). It is also preferred that the chemical treatment is conducted after embedding the DNA in agarose. It is also and additionally preferred that in the chemical treatment, a reagent that denatures the DNA duplex and/or a radical trap is (are) present.

It is also preferred that reporter molecules indicate the amplification either by an increase or a decrease in the fluorescence. It is particularly advantageous that the increase or the decrease in the fluorescence also is used directly for the analysis and a conclusion of the methylation status of the DNA to be analyzed is made from the fluorescent signal.

In turn, this can be achieved in different ways known to the person skilled in the art. First of all, it is possible to provide the probe oligonucleotides that bind adjacent to one another with different fluorescent dyes.

Either one of the dyes, insofar as it is found in the spatial vicinity to the other one and the other one is stimulated, can be stimulated to fluorescence by fluorescence [resonance] energy transfer (FRET). It is also possible, on the other hand, that one dye suppresses the fluorescence of the other dye, if it is adjacent spatially to the latter (quenching). Both methods can be utilized for visualization of the progress of the MLA. Logically, the methods are utilized in PCR as Taqman or Lightcycler assays.

It is further preferred according to the invention that the background DNA is present in 100-fold the concentration in comparison to the DNA to be investigated. It is further preferred that the background DNA is present in 1000× the concentration in comparison to the DNA to be investigated.

It is further preferred that the analysis or the additional analysis is optionally conducted by means of hybridization to oligomer arrays, whereby oligomers can be nucleic acids or molecules such as PNAs (Peptide Nucleic Acids) that are similar in their hybridization properties.

It is also preferred according to the invention that the analysis or, optionally, the further analysis is conducted by measuring the length of the amplified coupled probe oligonucleotides, whereby methods for length measurement comprise gel electrophoresis, capillary gel electrophoresis, chromatography (e.g. HPLC), mass spectrometry and other suitable methods.

It is advantageous that the amplificates themselves are provided with a detectable label for the detection. It is also advantageous that the labels are fluorescent labels or/and that the labels are radionuclides or/and that the labels are removable mass labels, which are detected in a mass spectrometer.

It is additionally preferred that the amplificates bear labels, such as biotin, for example, so that they can be selectively bound to solid phases. In a particularly preferred variant of the method, an oligonucleotide probe labeled with biotin as well as a fluorescently-labeled oligonucleotide probe are coupled together and then the products are bound to streptavidin, for example. A fluorescent signal of the bound species accordingly can only be measured if a coupling has occurred. The fluorescent signal is proportional to the number of ligations that have occurred within limits imposed by the method.

It is also [preferred] according to the invention that the amplificates are detected overall in the mass spectrometer and are thus clearly characterized by their mass.

Another subject of the present invention is also the use of a method according to the invention for the diagnosis and/or prognosis of adverse events for patients or individuals, whereby these adverse events belong to at least one of the following categories: undesired drug interactions; cancer diseases; CNS malfunctions, damage or disease; symptoms of aggression or behavioral disturbances; clinical, psychological and social consequences of brain damage; psychotic disturbances and personality disorders; dementia and/or associated syndromes; cardiovascular disease, malfunction and damage; malfunction, damage or disease of the gastrointestinal tract; malfunction, damage or disease of the respiratory system; lesion, inflammation, infection, immunity and/or convalescence; malfunction, damage or disease of the body as a consequence of an abnormality in the development process; malfunction, damage or disorder of the skin, the muscles, the connective tissue or the bones; endocrine and metabolic malfunction, damage or disease; headaches or sexual malfunction.

The use of a method according to the invention is also advantageous for distinguishing cell types or tissues or for investigating cell differentiation.

The subject of the present invention is also a kit comprised of a reagent containing bisulfite, labeled oligonucleotide probes, a preferably heat-stable ligase and buffers, as well as, optionally, instructions for conducting an assay according to the invention.

The preferred method consists of several steps, which can be summarized as follows: First, a DNA serum and/or other body fluids is (are) sampled from the patient and the DNA found therein is isolated if necessary. Then a chemical treatment, preferably with a bisulfite (=hydrogen sulfite, disulfite) is conducted, wherein, for example, all unmethylated cytosine bases are converted to uracil, but the methylated cytosine bases (5-methylcytosine) remain unchanged. In the second step of the method, an amplifying ligation is now conducted, in which, preferably the DNA to be investigated is amplified, but the background DNA is not, or is amplified only to a lesser extent. In any case, the amplification is produced, however, as a function of whether a specific methylation status is present on at least one DNA fragment in the sample, such as, for example, preferably all CpG positions methylated in the positions on the probe oligonucleotides. In the following, third step, the amplified fragments are now identified and conclusions are made on the methylation status in the genomic DNA sample. The presence of a disorder or another medical condition of the patient is preferably concluded from this.

The genomic DNA used in the method is preferably obtained from a DNA sample, whereby sources for DNA include, e.g., cell lines, blood, sputum, stool, urine, serum, cerebrospinal fluid, tissue embedded in paraffin, for example tissue from eyes, intestine, kidney, brain, heart, prostate, lungs, breast or liver, histological slides and all possible combinations thereof. The isolation of DNA from body fluids of an individual, such as sputum, serum, plasma, whole blood, urine or ejaculate, is particularly preferred.

A purification or concentration of the DNA is performed in several cases prior to the bisulfite treatment in order to avoid a disruption of the bisulfite reaction and/or the subsequent PCR by too high a content of impurities. It is known, however, that, for example, a PCR can be conducted from tissue, for example, after treatment with proteinase K without further purification, and this is true in a general sense also for the bisulfite treatment and subsequent PCR.

The chemical treatment is preferably conducted by treatment with a bisulfite (=hydrogen sulfite, disulfite), preferably sodium bisulfite (ammonium bisulfite is less suitable.) The reaction is either conducted according to a published variant, and preferably the DNA is embedded in agarose, in order to keep the DNA in the single-stranded state during treatment, or, however, according to a new variant, by treatment in the presence of a radical trap and a denaturing reagent, preferably an oligoethylene glycol dialkyl ether or, for example, dioxane. Prior to the PCR reaction, the reagents are removed either by washing in the case of the agarose method or by a DNA purification method (prior art, precipitation or binding to a solid phase, membrane) or, however, they are brought to a concentration range which no longer significantly influences the PCR simply by dilution.

It is now essential for the second method step that the methylation positions to be investigated are selected and suitable probe oligonucleotides are also selected, which permit the selective amplification of the DNA to be investigated. The selection of the positions is thus made either according to the premise that they should distinguish as much as possible between the methylation of the background DNA and that of the DNA to be investigated, or even the presence of such methylation in a large portion of the sample DNA can lead to the conclusion that a disorder or another specific medical condition is present in an individual. For this purpose, first, the methylation profiles are determined for the segments of a gene that are in question each time, both for the DNA to be investigated from individuals with disorders as well as also for the background DNA of healthy individuals. Those positions, which have the greatest differences between the DNA to be investigated and background DNA (for example in serum), will be selected as positions to be investigated. Such positions are already known for a plurality of genes, for example, for GSTpi, for HIC-1 and MGMT (vonWronski M A, Harris LC, Tano K, Mitra S, Bigner D D, Brent T P. (1992) Cytosine methylation and suppression of O6-methylguanine-DNA methyltransferase expression in human rhabdomyosarcoma cell lines and xenografts. Oncol Res.;4(4-5):167-74; Esteller M, Toyota M, Sanchez-Cespedes M, Capella G, Peinado M A, Watkins D N, Issa J P, Sidransky D, Baylin S B, Herman J G. (2000), Inactivation of the DNA repair gene O6-methylguanine-DNA methyltransferase by promoter hypermethylation is associated with G to A mutations in K-ras in colorectal tumorigenesis. Cancer Res. May 1;60(9):2368-71).

It is obvious that even in this case, the formation of an amplificate can provide sufficient information, since the situation is present, as it is also in MSP, such that the group of CpG positions is unmethylated practically up to 100%, for example, in the background DNA, but is methylated in the DNA to be investigated (which then only occurs in individuals with the disorder). If, in the MLA, one now uses probe oligonucleotides, which preferably bind to the sequence which forms in the bisulfite treatment from unmethylated background DNA, then only one ligation product is formed, if at least a small quantity of the DNA to be investigated is present overall.

It is also particularly preferred to conduct the method in a multiplexed manner for several methylation positions simultaneously in one batch. In this case, for example, preferably 4 different groups of CpG positions are investigated for their methylation. Commercial equipment for real-time PCR (e.g. ABI Prism) can distinguish 4 fluorescent dyes and is thus very well suitable for conducting 4× multiplexed MLA assays.

In the simplest case, the amplificates that form are now directly detected. For this purpose, all possible known molecular biology methods are considered, such as gel electrophoresis, sequencing, liquid chromatography or hybridizations.

Detection techniques, which are also suitable for the detection of the amplificates, are hybridization to oligomer arrays and, for example, primer extension (minisequencing) reactions. Hybridization to oligomer arrays can be used without further modification of protocols when compared with the closest prior art (Olek A, Olek S, Walter J; WO Patent 99/28498 A1). In this case, the amplificate or the amplificates are particularly preferably labeled fluorescently or radioactively or with removable mass tags, so that after the hybridization, the fragments bound to both oligonucleotides of a pair can be detected and quantified on the basis of this label. A plurality of amplificates can be detected simultaneously on such an oligomer array, so that such a method should be especially suited for the analysis of highly multiplexed MLAs. It is meaningful and preferred that the array also contains oligomers that do not bind to CpG positions for control of the experiment. These bind to ligation products of probe oligonucleotides that are not methylation-sensitive and that serve for quality control and/or quantification of the sample DNA.

A particularly preferred variant of the method, however, is the use of Taqman or Lightcycler technology variants for real-time detection. This can be achieved by a number of methods from the change in fluorescence that occurs during the amplification and is dependent on the methylation status. First of all, probe oligonucleotides can be used which bind specifically either to a sequence which is produced by chemical treatment from a DNA that is unmethylated at the corresponding position, or vice versa, to a sequence, which is produced by chemical treatment from a methylated DNA at the corresponding position. As stated above, these probes must hybridize adjacent to one another for the ligation. These probes are particularly preferably provided with two different fluorescent dyes: a quencher dye and a fluorescent dye serving as a marker. If an MLA reaction now occurs with the DNA to be investigated as the template, the quencher dye and the fluorescent dye serving as the marker are brought into contact with one another by ligation of the two probes. In this way, a decrease in the fluorescence of the marker dye is directly visible.

Different fluorescent dyes with different emission wavelengths of several probes are preferably utilized together with different quencher probes in order to be able to distinguish among the probes and thus to achieve a multiplexing.

If a more precise quantification of the degree of methylation of a methylation position is desired, then two pairs of probes competing with one another and having different dyes can also be utilized preferably, whereby one of these again preferably hybridizes in the case of an unmethylated position in the DNA to be investigated, while vice versa, the other preferably binds in the case of a methylated position. The methylation degree of the investigated position can then again be concluded from the ratio of the increases in fluorescence for the two dyes.

A basically different method, in which, however, there is also a change in fluorescence during the PCR, is known presently as LightCycler™ technology. The fact is utilized here that a fluorescence resonance energy transfer (FRET) can only occur between two dyes, if these are found in the immediate vicinity to one another, i.e., within 1-5 nucleotides. Only then can the second dye be excited by the emission of the first dye, and then in its turn, emit light of another wavelength, which is then detected. This method can be applied analogously also to MLA, except that in this case, the two probes are coupled after the ligation and can no longer be separated in the subsequent denaturing step.

In the present case of methylation analysis, a hybridization of a fluorescently labeled probe to the respective chemically treated DNA at a CpG position occurs, whereby the binding of this probe depends in turn on whether the DNA to be investigated was methylated or unmethylated at this position. Another probe with another fluorescent dye binds directly adjacent to this probe. This binding preferably occurs in turn as a function of methylation, if another methylatable position is present in the respective sequence segment. During the amplification, the DNA is now propagated, for which reason continuously more fluorescently labeled probes hybridize adjacent to the position in question and thus are coupled with one another, as long as the necessary methylation status for this was present, and thus an increasing FRET is measured.

Each of the oligonucleotide probes used particularly preferably hybridizes to a sequence which contained at least two CpG dinucleotides prior to the treatment according to step 1 of the method according to the invention. It is again particularly preferred to design the probes in such a way that as many such CG positions as possible lie in the sequence segment to which the two oligonucleotide probes hybridize.

A multiplexing with several different fluorescently labeled probes preferably is also produced by this method. It is particularly preferred again here that one of the probes that hybridize adjacent to one another contains a specific label each time, such as, for example, a quencher dye, and the other one contains another label that depends on sequence and is specific for the respective sequence, such as, for example, a fluorescent dye. It is thus possible and preferred, for example, that only one quencher dye is utilized in a multiplexed assay, but there are four different fluorescent dyes.

It is possible to use two fluorescent dyes, whereby one of them can stimulate the fluorescence of the other, or to use one fluorescent dye and one quencher dye, which can correspondingly extinguish the fluorescence of the other dye.

The two methods differ in result principally by the fact that in one case a decrease in fluorescence is measured, whereas an increase is measured in the other case, during the amplification.

In another particularly preferred variant of the method, in addition to the ligation step, there occurs an extension of at least one oligonucleotide probe, which further increases the specificity of the amplification method. In this case, the oligonucleotide probes hybridize not directly adjacent to one another, but at a small distance, most preferably 1-10 bases distant from one another. This gap is filled with nucleotides by a polymerase reaction. This extension by means of an additionally utilized polymerase can either be produced in a methylation-specific manner, if a CpG dinucleotide is present in the as-yet untreated DNA at the respective position, or may be used only to increase the sequence specificity. The extension is preferably produced via either only one base or a relatively small number of bases, most preferably between 1 and 10 bases. In a particularly preferred variant of the method, the oligonucleotide probes directly delimit the CG position to be investigated. One of the oligonucleotide probes most preferably overlaps with one base of the CG dinucleotide. It is again particularly preferred that only one methylatable position is found in the segment between the oligonucleotide probes, which is filled by the primer extension.

An oligonucleotide probe with known sequence of n nucleotides is thus extended with a heat-stable polymerase by at most the number of nucleotides that lie between the 3′-end of the first oligonucleotide probe and the 5′-end of the second hybridized oligonucleotide probe. Preferably, at least one nucleotide bears a detectable label. This detectable label can in turn interact most preferably with another label, which is bound to one of the oligonucleotide probes, so that the extent of the incorporation of the labeled nucleotide can be measured. This interaction is most preferably fluorescence resonance energy transfer (FRET). In a particularly preferred variant of the method either the first oligonucleotide probe and/or the second oligonucleotide probe thus bears a detectable label. The type of extension here preferably depends on the methylation status of at least one cytosine in the genomic DNA sample, or, however, on possibly present SNPs, point mutations or deletions, insertions and inversions.

In a preferred variant of the method, the utilized nucleotides are chain-terminating nucleotides and/or chain-lengthening nucleotides. The terminating nucleotide is preferably a 2′,3′-dideoxynucleotide and the chain-lengthening nucleotide is a 2′-deoxynucleotide. It is particularly preferred that a terminating nucleotide is incorporated, which does not permit subsequent ligation, if the methylation status typical for the background DNA was present in the respective template strand prior to the treatment according to step 1 of the method. A chain-lengthening nucleotide is incorporated, in contrast, if the methylation status typical for the DNA to be investigated was present in the respective template strand prior to the treatment according to step 1 of the method.

In another particularly preferred variant of the method, not all four nucleotides are utilized for the amplification, but only a maximum of three nucleotides, most preferably either the nucleotides dATP, dCTP and dTTP or the nucleotides dATP, dGTP and dTTP. Alternatively dUTP can be utilized each time instead of dTTP.

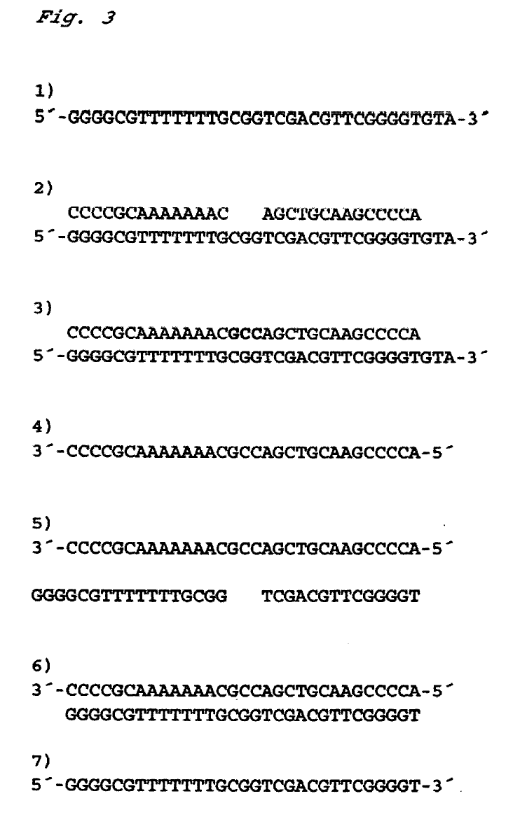

A sequence example for the application of only three nucleotides is shown in FIG. 3.

Most preferably, when the ligase reaction is combined with a polymerase step, at least one of the oligonucleotides is modified in such a way that it cannot be extended at the 3′-end by the polymerase. The 3′-end most preferably is present functionalized with a phosphate group or modified with 2′,3′-dideoxy.

It is again particularly preferred that the polymerase utilized has no 5′-exonuclease activity or only a very slight activity. The Stoffel fragment of the Taq polymerase is most preferably used.

In another particularly preferred variant of the method, at least one blocker oligonucleotide is utilized in addition to the oligonucleotide probes. This blocker oligonucleotide preferably binds to the background DNA and prevents the ligase reaction and/or primer extension in the case of an additional polymerase step.

In a particularly preferred variant of the method, a blocker oligonucleotide binds to positions which are also covered by one of the oligonucleotide probes. In another particularly preferred variant of the method, a blocker oligonucleotide binds to positions which are partially covered by the first oligonucleotide probe and partially by the second oligonucleotide probe. In this case, a blocker oligonucleotide binds, among others, to the position at which a ligation of the hybridized probe oligonucleotides otherwise could occur.

In another particularly preferred variant of the method, blocker oligonucleotides bind to the positions between the two hybridized oligonucleotide probes, which otherwise could be occupied by the extension of the first probe by means of a polymerase reaction.

When blocker oligonucleotides are used, it is particularly preferred that they are present modified in such a way that they cannot be extended at the 3′-end by the polymerase. The 3′-end is most preferably functionalized with a phosphate group or modified by 2′,3′-dideoxy. The analogous use of PNA (Peptide Nucleic Acids) or other nucleic acid analogs as blocker molecules is also particularly preferred.

It is also preferred that the blockers cannot be basically decomposed by the 5-exonuclease activity of a possibly utilized polymerase. For this purpose, the 5′-ends of the blockers can be present modified, for example, or, however, most preferably, one or more phosphorothioate bridges can be present toward the 5′-end of the blocker oligonucleotide.

The probe oligonucleotides are most preferably phosphorylated prior to their use in the MLA or are phosphorylated at the 5′-end directly by means of conventional oligonucleotide synthesis. The probes are most preferably phosphorylated by means of polynucleotide kinase and ATP. The phosphorylation is only necessary for the second oligonucleotide probe in each case.

In summary a method is particularly preferred for the detection of cytosine methylation in DNA samples, in which the following steps are conducted:
1. A genomic DNA sample is treated in such a way that the unmethylated cytosine bases are converted to uracil, while the 5-methylcytosine bases remain unchanged,
2. the chemically treated DNA sample is amplified with the use of at least 2 pairs of essentially complementary probe oligonucleotides as well as a ligase, and
3. the amplificates are analyzed and the methylation status in the DNA to be investigated is concluded from the presence of an amplificate.

In a particularly preferred method variant, the sample DNA is obtained from serum or other body fluids of an individual. It is also preferred that the DNA samples are obtained from cell lines, blood, sputum, stool, urine, serum, cerebrospinal fluid, tissue embedded in paraffin, for example, tissue from eyes, intestine, kidney, brain, heart, prostate, lungs, breast or liver, histological slides and all possible combinations thereof.

In a particularly preferred variant of the method, the chemical treatment is conducted with a bisulfite (=disulfite, hydrogen sulfite). It is preferred that the chemical treatment is conducted after embedding the DNA in agarose. It is also preferred that a reagent that denatures the DNA duplex and/or a radical trap is (are) present in the chemical treatment.

A Taqman assay is most preferably conducted for the analysis. It is also preferred that a LightCycler assay (as described above) is conducted.

It is particularly preferred that the oligonucleotides used in addition to the primers do not make available a 3′-OH function. Also, the reporter oligonucleotides most preferably bear at least one fluorescent label.

It is particularly preferred that the reporter molecules indicate the amplification either by an increase or a decrease in the fluorescence and that the increase or decrease in fluorescence also is used directly for the analysis and a conclusion on the methylation status of the DNA to be analyzed is made from the fluorescent signal.

A method is also particularly preferred, in which the further analysis is conducted by measuring the length of the amplified DNA to be investigated, whereby methods for length measurement comprise gel electrophoresis, capillary gel electrophoresis, chromatography (e.g. HPLC), mass spectrometry and other suitable methods.

A method is also particularly preferred, in which the further analysis is conducted by sequencing, whereby methods for sequencing comprise the Sanger method, the Maxam-Gilbert method, and other methods such as sequencing by hybridization (SBH). A method is also preferred, wherein the sequencing (according to Sanger) is designed for each CpG position or a small group of CpG positions, each with a separate primer oligonucleotide and the extension of the primer constitutes only one or just a few bases and the methylation status of the respective positions in the DNA to be investigated is concluded from the type of primer extension.

In a particularly preferred variant of the method, a conclusion is made on the presence of a disease or another medical condition of the patient from the methylation degree at the different CpG positions investigated.

It is particularly preferred that the amplificates themselves are also provided with a detectable label for the detection. The labels preferably involve fluorescent labels, radionuclides, or removable mass labels, which are detected in a mass spectrometer.

A variant of the method is also preferred, wherein the amplificates are detected overall in the mass spectrometer and are thus clearly characterized by their mass.

Another subject of the present invention is the use of one of the described methods for the diagnosis and/or prognosis of adverse events for patients or individuals, whereby these adverse events belong to at least one of the following categories: undesired drug interactions; cancer diseases; CNS malfunctions, damage or disease; symptoms of aggression or behavioral disturbances; clinical, psychological and social consequences of brain damage; psychotic disturbances and personality disorders; dementia and/or associated syndromes; cardiovascular disease, malfunction and damage; malfunction, damage or disease of the gastrointestinal tract; malfunction, damage or disease of the respiratory system; lesion, inflammation, infection, immunity and/or convalescence; malfunction, damage or disease of the body as a consequence of an abnormality in the development process; malfunction, damage or disorder of the skin, the muscles, the connective tissue or the bones; endocrine and metabolic malfunction, damage or disease; headaches or sexual malfunction.

The use of one of the described methods is also preferred for distinguishing cell types or tissues or for investigating cell differentiation.

The following examples explain the invention:

### EXAMPLE 1

Preparation of Unmethylated and Methylated DNA and Bisulfite Treatment

For the preparation of methylated DNA, human genomic DNA was treated with S-adenosylmethionine and CpG methylase (SssI, New England Biolabs) according to the information of the manufacturer. The preparation of unmethylated DNA was not necessary as a reference for the following Examples, since the respective positions are unmethylated without exception in commercially available human DNA (Promega). The bisulfite treatment was conducted according to the published agarose method (Olek A, Oswald J, Walter J. A modified and improved method for bisulphite based cytosine methylation analysis. Nucleic Acids Res. 1996 Dec. 15;24(24):5064-6). Methylated DNA and untreated DNA were utilized in identical amounts (approximately 700 ng) in each of two different, but analogously conducted bisulfite reactions.

### EXAMPLE 2

The sequence GGGCGTTTTTTTGCGGTCGACGTTCGGGGTGTA (SEQ-ID:1) (after bisulfite treatment) was investigated by means of MLA. This sequence is present when the respective methylation positions in the DNA sample were methylated. The DNA sample treated with SssI and with bisulfite of Example 1 was used. The probe oligonucleotides GGCGTTTTTTTGCGG (SEQ-ID:2) and TCGACGTTCGGGGT (SEQ-ID:3) as well as the probe oligonucleotides complementary thereto CCGCAAAAAAACGCC (SEQ-ID:4) and ACCCCGMCGTCGA (SEQ-ID:5) were used, whereby SEQ-ID:3 and SEQ-ID:4 were each phosphorylated beforehand at the 5′-end by means of polynucleotide kinase Conditions such as those described in WO 94/08047 were used for the ligation (40 cycles).

The ligation products were detected by means of polyacrylamide-gel electrophoresis. In contrast, a control experiment with unmethylated control DNA according to Example 1 produced no detectable product.

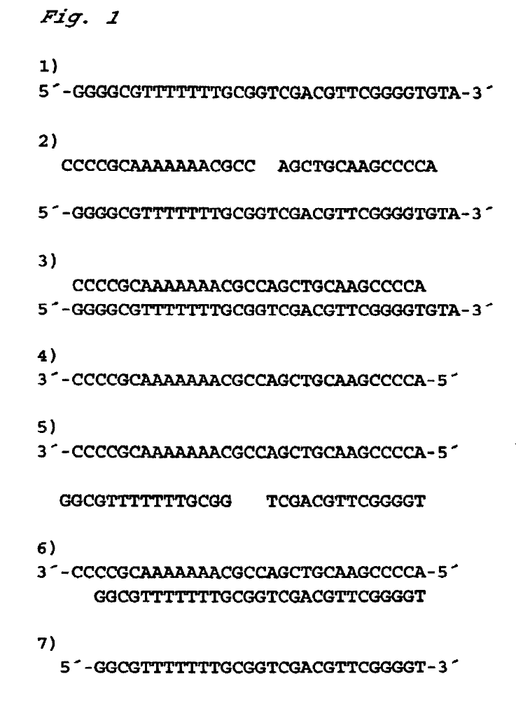

The MLA reaction is shown schematically in FIG. 1. After the bisulfite treatment, the DNA is present in single-stranded form (1) and permits, under suitable hybridization conditions, the hybridizing of the probes if the CG positions were methylated prior to the bisulfite reaction (2). The probe oligonucleotides are ligated (3). The double strand that forms is now denatured in the next step, so that the ligated probes can also serve in turn as the template (4). Complementary probe oligonucleotides now hybridize to this (5) and a repeated ligation occurs (6). After denaturing, the complementary single strand is now available again as the template and steps (2) to (7) can be repeated several times until sufficient ligation product has formed.

### EXAMPLE 3

MLA with the Use of a Blocker Oligonucleotide

Since larger quantities of background DNA can apparently contribute to false-positive results, a blocker for background DNA, as described above, can be additionally utilized. If the experiment is designed analogously to Example 2, then TGTGGTTGATGTTTG (SEQ-ID:6) can be used as the blocker. This blocker preferably binds when the background DNA was completely unmethylated in this region. Under these conditions, it is possible to detect methylated templates even in a ratio of 1:100 to 1:1000 depending on the total DNA concentration without risking false-positive results for the completely unmethylated control DNA.

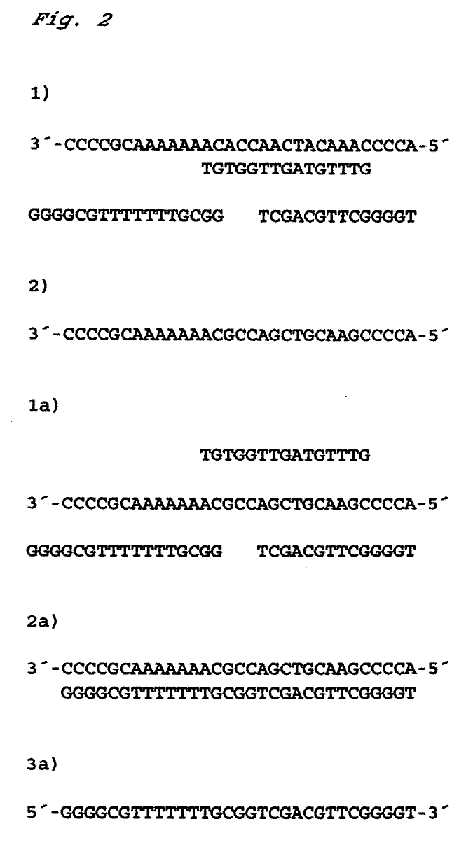

The use of a blocker oligonucleotide is shown in FIG. 2. The latter binds to the template DNA (1) and prevents the hybridization of the oligonucleotide probes. Thus after dehybridization only the template strand remains and a ligation does not occur.

If a template strand is utilized which corresponds to methylated DNA (1a), then a hybridization of the blocker oligonucleotide cannot occur. The hybridization of the probe oligonucleotides as well as their ligation occurs essentially without hindrance (2a). A ligation product (3a) is formed.

### EXAMPLE 4

Use of an MLA assay with additional polymerase reaction.

The sequence GGGCGTTTTTTTGCGGTCGACGTTCGGGGTGTA (SEQ-ID:1) (after bisulfite treatment) was investigated. This sequence is present when the respective methylation positions in the DNA sample were methylated. The DNA sample treated with SssI and with bisulfite of Example 1 was used. The probe oligonucleotides GGGGCGTTTTTTTGCGG (SEQ-ID:7) and TCGACGTTCGGGGT (SEQ-ID:3) as well as the probe oligonucleotides complementary thereto CAAAAAAACGCCCC (SEQ-ID:8) and ACCCCGAACGTCGA (SEQ-ID:5) were used, whereby all probe oligonucleotides were each phosphorylated beforehand at the 5′-end by means of polynucleotide kinase.

Additionally, Taq polymerase (Amplitaq) and nucleotides were [used]. Conditions such as those described in WO 94/08047 and EP 0 439,182 were used for the ligation.

The ligation products were detected in turn by means of polyacrylamide-gel electrophoresis.

In contrast, a control experiment with unmethylated control DNA according to Example 1 produced no detectable product.

In one variant of the method, not all nucleotides can be used. For example, in the above Example, only dGTP, dCTP and ddATP can be utilized as nucleotides. The ddATP is only incorporated if an unmethylated fragment is utilized unintentionally as the template in the polymerase reaction. In this case, however, since it serves as a chain terminator, a ligation no longer occurs.

The ligase/polymerase reaction is shown schematically in FIG. 3. After the bisulfite treatment, the DNA is present in single-stranded form (1) and permits, under suitable hybridization conditions, the hybridizing of the probes, if the CG positions were methylated prior to the bisulfite reaction. The gap between the probes is filled in a polymerase reaction and then the probes are ligated (3). The double strand that forms is now denatured in the next step, so that the ligated probes can also serve in turn as the template (4). Complementary probe oligonucleotides now hybridize to this (5) and a repeated ligation occurs (6). After denaturing, the complementary single strand is now available again as the template and steps (2) to (7) can be repeated several times until sufficient ligation product has formed.

1. A method for the detection of cytosine methylation in DNA samples, is hereby characterized in that the following steps are conducted:
a) a genomic DNA sample is treated in such a way that the unmethylated cytosine bases are converted to uracil, while the 5-methylcytosine bases remain unchanged;
b) the chemically treated DNA sample is amplified with the use of at least 2 pairs of essentially complementary probe oligonucleotides as well as a ligase and
c) the amplificates are analyzed and the methylation status in the DNA to be investigated is concluded from the presence of an amplificate.

2. The method according to claim 1, further characterized in that in the second step, the DNA to be investigated is preferred over the sequence-homologous background DNA as the template.

3. The method according to claim 1, further characterized in that the methylation status in the DNA to be investigated is concluded from the analysis of additional positions in the amplificate.

4. The method according to claim 1, wherein in step b), the probe oligonucleotides then hybridize to a template, if the CpG positions which are covered by these in the genomic DNA sample (or the DNA to be investigated) were methylated and wherein the same probe oligonucleotides hybridize essentially to a lesser extent to templates which were completely or partially unmethylated at these positions.

5. The method according to claim 1, wherein in step b), the probe oligonucleotides then hybridize to a template, if the CpG positions which are covered by these in the genomic DNA sample (or the DNA to be investigated) were unmethylated and wherein the same probe oligonucleotides hybridize essentially to a lesser extent to templates which were present completely or partially methylated at these positions.

6. The method according to claim 1, further characterized in that step b) is designed in detail as follows:
1) the probe oligonucleotides, which hybridized to adjacent positions on the template are coupled together by ligation,
2) the coupled probe oligonucleotides are dehybridized,
3) probe oligonucleotides complementary to the coupled probe oligonucleotides hybridize to the already coupled probe oligonucleotides and are coupled in turn by ligation and
4) the coupled probe oligonucleotides serve as a template for further ligation steps, so that a further propagation of the coupled probe oligonucleotides is produced.

7. The method according to claim 1, further characterized in that at least one of the probe oligonucleotides bears a phosphate group at the 5′-end.

8. The method according to claim 1, further characterized in that at least one of the probe oligonucleotides is provided with a label detectable by fluorescence.

9. The method according to claim 1, further characterized in that at least one of the probe oligonucleotides is provided with a detectable label.

10. The method according to claim 8 or 9, further characterized in that at least two probe oligonucleotides are provided with labels, and that these modify their properties as a function of the distance between them.

11. The method according to claim 10, further characterized in that the probe oligonucleotides bear at least one fluorescent label.

12. The method according to claim 11, further characterized in that the probe molecules indicate the amplification either by an increase or a decrease of the fluorescence.

13. The method according to claim 12, further characterized in that the increase or decrease in fluorescence also is used directly for the analysis and a conclusion on the methylation status of the DNA to be investigated is made from the modified fluorescent signal.

14. The method according to claim 1, further characterized in that the background DNA is present in 100× the concentration in comparison to the DNA to be investigated.

15. The method according to claim 1, further characterized in that the background DNA is present in 1000× the concentration in comparison to the DNA to be investigated.

16. The method according to claim 1, further characterized in that the DNA samples are obtained from serum or other body fluids of an individual.

17. The method according to claim 1, further characterized in that the DNA samples are obtained from cell lines, blood, sputum, stool, urine, serum, cerebrospinal fluid, tissue embedded in paraffin, for example, tissue from eyes, intestine, kidney, brain, heart, prostate, lungs, breast or liver, histological slides and all possible combinations thereof.

18. The method according to claim 1, further characterized in that step a) is conducted with a bisulfite (=disulfite, hydrogen sulfite).

19. The method according to claim 18, further characterized in that the chemical treatment is conducted after embedding the DNA in agarose.

20. The method according to claim 18, further characterized in that a reagent that denatures the DNA duplex and/or a radical trap is (are) present in the chemical treatment.

21. The method according to claim 1, further characterized in that the analysis according to step c) is made by means of hybridization to oligomer arrays, wherein oligomers can be nucleic acids or molecules such as PNAs that are similar in their hybridization properties.

22. The method according to claim 1, further characterized in that the analysis according to step c) is made by means of measuring the length of the amplified DNA to be investigated, whereby methods for length measurement comprise gel electrophoresis, capillary gel electrophoresis, chromatography (e.g. HPLC), mass spectrometry and other suitable methods.

23. The method according to claim 1, further characterized in that the analysis according to step c) is conducted by means of sequencing, whereby methods for sequencing comprise the Sanger method, the Maxam-Gilbert method, and other methods such as sequencing by hybridization (SBH).

24. The method according to claim 1, further characterized in that a conclusion is made on the presence of a disease or another medical condition of the patient from the methylation status at the different CpG positions investigated.

25. The method according to claim 1, further characterized in that the amplificates themselves are provided with a detectable label for the detection.

26. The method according to claim 25, further characterized in that the labels are fluorescent labels.

27. The method according to claim 25, further characterized in that the labels are radionuclides.

28. The method according to claim 25, further characterized in that the labels are removable mass labels which are detected in a mass spectrometer.

29. The method according to claim 25, further characterized in that the amplificates are detected as a whole in the mass spectrometer and are thus clearly characterized by their mass.

30. The method according to claim 1, further characterized in that in addition to the probe oligonucleotides, a blocker oligonucleotide is utilized, which preferably binds to the background DNA and prevents the hybridization of the probe oligonucleotides to the background DNA.

31. The method according to claim 30, further characterized in that two blocker oligonucleotides (or blocker PNAs, generally blocker molecules) that are complementary to one another are used.

32. The method according to claim 31, further characterized in that the blocker molecules preferably bind to template strands, which correspond in their sequence to a DNA that is methylated after treatment according to step a).

33. The method according to claim 31, further characterized in that the blocker molecules preferably bind to template strands, which correspond in their sequence to a DNA that is unmethylated after treatment according to step a).

34. The method according to claim 31, further characterized in that the blocker molecules bind to several CpG positions in the template DNA.

35. The method according to claim 31, further characterized in that the blocker molecules bind to several TpG or CpA positions in the template DNA.

36. The method according to claim 31, further characterized in that the blocker oligonucleotides are modified at their 3′-end and cannot be essentially decomposed by a polymerase with nuclease activity.

37. The method according to claim 1, further characterized in that step b) is designed in detail as follows:
1) the probe oligonucleotides (probes) hybridize to positions on the template strand in such a way that a gap of at least one base remains between the 3′-end of the first probe and the 5′-end of the second probe,
2) the 3′-end of the first probe is extended by a polymerase reaction, wherein nucleotides complementary to the template strand are incorporated each time,
3) the elongated first probe is coupled by ligation to the elongated second probe,
4) the coupled probe oligonucleotides are dehybridized,
5) probe oligonucleotides complementary to the coupled probe oligonucleotides hybridize to the already coupled probe oligonucleotides and are coupled in turn by ligation and
6) the coupled probe oligonucleotides serve as a template for further ligation steps, so that a further propagation of the coupled probe oligonucleotides is produced.

38. The method according to claim 37, further characterized in that step 5) is also conducted analogously to steps 1)-3).

39. The method according to one of claims 37 or 38, further characterized in that a heat-stable polymerase is used.

40. The method according to claim 1, further characterized in that a heat-stable ligase is used.

41. The method according to claim 1, further characterized in that several sets of oligonucleotide probes are utilized for several groups of methylation positions and thus a multiplexing of the assay is achieved.

42. Use of a method according to claim 1 for the diagnosis and/or prognosis of adverse events for patients or individuals, whereby these adverse events belong to at least one of the following categories: undesired drug interactions; cancer diseases; CNS malfunctions, damage or disease; symptoms of aggression or behavioral disturbances; clinical, psychological and social consequences of brain damage; psychotic disturbances and personality disorders; dementia and/or associated syndromes; cardiovascular disease, malfunction and damage; malfunction, damage or disease of the gastrointestinal tract; malfunction, damage or disease of the respiratory system; lesion, inflammation, infection, immunity and/or convalescence; malfunction, damage or disease of the body as a consequence of an abnormality in the development process; malfunction, damage or disease of the skin, the muscles, the connective tissue or the bones; endocrine and metabolic malfunction, damage or disease; headaches or sexual malfunction.

43. Use of a method according to claim 1 for the differentiation of cell types or tissues or for the investigation of cell differentiation.

44. A kit comprising a reagent containing bisulfite, labeled oligonucleotide probes, a preferably heat-stable ligase and buffers, as well as, optionally, instructions for conducting an assay according to the invention.
